(19) United States
(12) Patent Application Publication
(10) Pub. No.: US 2024/0207169 A1
(43) Pub. Date: Jun. 27, 2024

(54) COMPOSITION FOR IMPROVING SKIN WRINKLE, MOISTURIZING, STRENGTHENING SKIN BARRIER, AND PROMOTING SKIN REGENERATION, COMPRISING MIXED LACTIC ACID BACTERIA AS ACTIVE INGREDIENT

(71) Applicant: PFNATURE CO., LTD., Gyeonggi-do (KR)

(72) Inventor: Ji Hye YANG, Gyeonggi-do (KR)

(21) Appl. No.: 17/915,661

(22) PCT Filed: May 19, 2022
(86) PCT No.: PCT/KR2022/007175

§ 371 Date: Jul. 13, 2023

(30) Foreign Application Priority Data
Jan. 10, 2022 (KR) 10-2022-0003158

(51) Int. Cl. A61K 8/99; A23L 33/135; A61Q 19/08

(52) U.S. Cl. CPC A61K 8/99; A23L 33/135; A61Q 19/08

(57) ABSTRACT

A composition includes mixed bacterial strains of Bacillus sp. pfbarrier 01 (KCTC 14628BP), Lactobacillus pentosus pfbio035 (KCTC 14684BP) and Lactobacillus sakei subsp. sakei pfbio005 (KCTC 14130BP), a culture solution thereof, or a compound extract thereof as an active ingredient to offer beneficial activities in improving skin wrinkles, moisturizing skin, strengthening skin barriers, and promoting regeneration of wounded skin. The composition poses non-cytotoxicity and high beneficial effects to improve skin wrinkles, moisturize skin, strengthen skin barriers, and help regeneration of wounded skin, which makes it highly applicable to the fields of foods and cosmetics. Besides, the composition is not only very safe for the human body but also has high stability.

### CROSS REFERENCE TO RELATED APPLICATION (S) AND CLAIM OF PRIORITY

This application claims benefit under 35 U.S.C. 119, 120, 121, or 365 (c), and is a National Stage entry from International Application No. PCT/KR2022/007175, filed May 19, 2022, which claims priority to the benefit of Korean Patent Application No. 10-2022-0003158 filed in the Korean Intellectual Property Office on Jan. 10, 2022, the entire contents of which are incorporated herein by reference.

### BACKGROUND

### 1. Field of the Invention

The present invention relates to a composition comprising a combination of assorted lactic acid bacteria as an active ingredient, and particularly to a composition comprising three mixed bacterial strains of Bacillus sp. pfbarrier 01 (KCTC 14628BP), Lactobacillus pentosus pfbio035 (KCTC 14684BP) and Lactobacillus sakei subsp. sakei pfbio005 (KCTC 14130BP), a culture solution thereof, or a compound extract thereof as an active ingredient and offering beneficial activities to improve skin wrinkles, moisturize skin, strengthen skin barriers, and promote regeneration of wounded skin.

### 2. Description of the Related Art

Some of the substances that play an important role in maintaining the homeostasis of organisms are a variety of bioactive substances derived from the organisms. Many research studies have been conducted on a number of bioactive substances. Among others, researches on the biologically active substances isolated from microorganisms, plants or animals, are of great importance in the fields of bioscience and medicine.

Examples of substances known to have beneficial effects in improving skin wrinkles, skin whitening and exert antioxidant activities are the conventional vitamin C, retinoic acid, transforming growth factors (TGFs), and the like. But, substances such as vitamin C, retinoic acid, TGFs, animal placenta-derived protein (JP8-231370), betulinic acid (JP8-208424), and chlorella extracts (JP9-40523, JP10-36283, promoting proliferation of fibroblasts) have limitations in their uses due to safety issue posing a risk of irritations and redness when applied to the skin and make too little effect to provide substantially beneficial effects in improving skin elasticity and wrinkles, helping skin whitening, etc.

Moreover, it is the latest trend to introduce natural product-derived bioactive substances into cosmetics to strengthen the innate defense of the skin and induce the functional improvement of the cosmetics on the skin. And, there is an urgent demand for development of natural materials that ensure safety for human body and stability of active ingredients and most importantly offer stronger effects than the existing substances in improving skin wrinkles, moisturizing skin, and helping regenerate wounded skin.

Besides, cosmetic materials need to be balanced in safety, efficacies and physical properties. Economic feasibility is another crucial factor in developing cosmetic materials. Hence, cosmetic materials are developed using various developing methods, such extraction of natural products and as microorganism-induced fermentation, in order to optimize skin permeability, anti-aging functions, bioactivities, safety, and properties. As the boundaries between cosmetics and foods and pharmaceuticals are blurring, a variety of cosmeceutical products that combine the stability of cosmetics and the effectiveness of pharmaceuticals and health functional foods continue to grow rapidly.

Under this background, the inventors of the present invention have been conducting research on the combination of lactic acid bacteria with effects of improving skin conditions, such as improving skin wrinkles or moisturizing skin. As a result of research studies to find out a combination of lactic acid bacteria with effects to improve various skin conditions, the present inventors have confirmed that a combination of three bacterial strains isolated from foods such as beans or kimchi and identified exhibits high activities in improving skin wrinkles, strengthening skin barriers and enhancing skin regeneration and thus can be used for the purpose of improving skin conditions, thereby completing the present invention.

### SUMMARY

It is therefore an object of the present invention to provide a composition comprising three mixed bacterial strains of Bacillus sp. pfbarrier 01, Lactobacillus pentosus pfbio035 and Lactobacillus sakei subsp. sakei pfbio005, a culture solution thereof, or a compound extract thereof that exhibit activities to improve skin wrinkles, moisturize skin, strengthen skin barriers, and enhance regeneration of wounded skin.

Other objects and advantages of the invention will become apparent to those skilled in the art from the following description of embodiments.

In one aspect of the present invention, there is provided a cosmetic composition comprising three mixed bacterial strains of Bacillus sp. pfbarrier 01 (KCTC 14628BP), Lactobacillus pentosus pfbio035 (KCTC 14684BP) and Lactobacillus sakei subsp. sakei pfbio005 (KCTC 14130BP), a culture solution thereof, or a compound extract thereof as an active ingredient.

The inventors of the present invention have been delving into a variety of natural materials in regard to their effects of improving skin conditions and tried to find a combination of lactic acid bacteria highly effective in improving skin conditions. As a result, the present inventors have demonstrated that a culture solution of three mixed bacterial stains of Bacillus sp. pfbarrier 01, Lactobacillus pentosus pfbio035 and Lactobacillus sakei subsp. sakei pfbio005 or a compound extract thereof makes synergistic effects in improving skin wrinkles and moisturizing skin and high beneficial effects in strengthening skin barriers and enhancing regeneration of wounded skin as well and causes no issue of safety when applied to the skin, thereby completing the present invention.

The Bacillus sp. pfbarrier 01 strain is internationally deposited under accession number KCTC14628BP with the Korean Collection for Type Culture (KCTC) of the Korea Research Institute of Bioscience and Biotechnology designated as an International Depository Authority (IDA) under Budapest Treaty. The Lactobacillus pentosus pfbio035 strain and the Lactobacillus sakei subsp. sakei pfbio005 strain are also internationally deposited with the KCTC under accession number KCTC14684BP and KCTC 14130BP, respectively. In this disclosure, the terms “Bacillus sp. pfbarrier 01 strain”, “pfbarrier 01 strain” and “pfbarrier 01” can be used interchangeably with the same meaning; the terms “Lactobacillus pentosus pfbio035 strain”, “pfbio035 strain” and “pfbio035” can be used interchangeably with the same meaning; and the terms “Lactobacillus sakei subsp. sakei pfbio005 strain”, “pfbio005 strain” and “pfbio005” can be used interchangeably with the same meaning.

The term “extract” as used herein includes an extract itself, including an extract solution obtained from a culture solution of lactic acid bacteria by an extraction process, a dilute solution or concentrated solution of the extract solution, a dried form of the extract solution obtained by drying, a modified or purified form of the extract solution, or a mixture thereof; and an extract of any formulation that can be formed from the extract solution. Preferably, the extract of the present invention may be prepared in a dry powder form after extraction before being put into use. In extracting the culture solution of the three mixed strains, the extraction method used for preparation of the extract is not specifically limited and may include any extraction method commonly used in the related art. Non-limiting examples of the extraction method may include hot water extraction, ultrasonic extraction, filtration extraction, reflux extraction, etc., which may be adopted alone or in combination.

The extraction solvent used to extract the culture solution of the three mixed strains is not specifically limited and may include any solvent known in the related art. Non-limiting examples of the extraction solvent may include water; C1-C4 lower alcohols, e.g., methanol, ethanol, propyl alcohol, or butyl alcohol; polyhydric alcohols, e.g., glycerin, butylene glycol or propylene glycol; and hydrocarbon-based solvents, e.g., methyl acetate, ethyl acetate, acetone, benzene, hexane, diethyl ether, or dichloromethane; or a mixture thereof. Preferably, the extraction solvent is water, lower alcohols, 1,3-butylene glycol, or ethyl acetate, which may be used alone or in combination.

In order to remove floating solid particles, the extract obtained by hot water extraction or cold extraction may be filtered, for example, by filtration with nylon or the like or cryofiltration to filter out the floating particles. The particle-free extract may be put into use immediately, or dried out through freeze drying, hot air drying or spray drying before being used.

The cosmetic composition of the present invention is characterized in that it inhibits the expression of matrix metalloproteinase-1 (MMP-1) gene and increases the expression of type 1 procollagen gene to improve skin wrinkles. In a preferred embodiment of the present invention, a treatment with the culture extract of the three mixed strains according to the present invention resulted in reducing the protein expression of MMP-1 (FIG. 2) in a concentration-dependent manner and increasing the gene expression of type 1 procollagen in a concentration-dependent manner (FIG. 3).

The cosmetic composition of the present invention is also characterized in that it promotes the synthesis of hyaluronan (hyaluronic acid) to moisture the skin. In a preferred embodiment of the present invention, it was confirmed that a treatment with the culture extract of the three mixed strains according to the present invention increased the production yield of hyaluronan in a concentration-dependent manner and that the compound extract made from the three lactic acid bacteria according to the present invention exhibited high synergistic effects in moisturizing the skin, relative to the single-strain extracts each made from one strain (FIG. 4).

The cosmetic composition of the present invention is also characterized in that it increases the expression of filaggrin gene to strengthen the skin barrier. The precursor of filaggrin is profilaggrin that is a protein constituting keratohyalin granules in the epidermal granular layer. Profilaggrin is broken down into filaggrin in the final differentiation process of keratinocytes. The filaggrin aggregates filaments in formation of cornified cellenvelopes to form a hard and flat structure of corneocytes and acts as a brick in the skin barrier. In a preferred embodiment of the present invention, it was confirmed that a treatment with the three-strain culture extract according to the present invention resulted in a significant increase in the mRNA expression of filaggrin (FIG. 5).

The cosmetic composition of the present invention is also characterized in that it promotes regeneration of wounded skin. In a preferred embodiment of the present invention, it was confirmed that a treatment with the three-strain culture extract according to the present invention significantly promoted regeneration of wounded skin (FIG. 6).

The cosmetic composition of the present invention is derived from an edible LAB (Lactic Acid Bacteria) material and hence not harmful to human body. The edible LAB material, when mixed in cosmetics, does not deteriorate the quality of the cosmetics, so it can be suitably used in cosmetic compositions. The LAB material is particularly desirable when used in functional cosmetic compositions due to its functions associated with improving skin wrinkles, moisturizing skin, strengthening skin barriers, and promoting regeneration of wounded skin.

The cosmetic composition of the present invention may be formulated into any selected one of formulations that may include, but are not limited to, skin softeners, skin toners, astringent, lotions, milk lotions, moisturizing lotions, nourishing lotions, massage creams, nourishing creams, moisturizing creams, hand creams, foundations, essences, nourishing essences, face mask packs, soaps, cleansing foams, cleansing lotions, cleansing creams, body lotions, and body cleansers.

The cosmetic composition of the present invention may further include at least one cosmetically acceptable carrier commonly blended in general skin care cosmetics and other conventional ingredients, examples of which carrier may include, but are not limited to, oils, water, surfactants, humectants, lower alcohols, thickeners, chelating agents, pigments, preservatives, and fragrances.

The cosmetically acceptable carrier included in the cosmetic composition of the present invention varies depending on the formulation type of the cosmetic composition.

The carrier component available in the present invention formulated as an ointment, paste, cream, or gel may include, but is not limited to, animal oils, vegetable oils, waxes, paraffins, starches, tragacanth, cellulose derivatives, polyethylene glycol, silicone, bentonite, silica, talc, or zinc oxide, which may be used alone or in combination.

The carrier component available in the present invention formulated as a powder or spray may include, but is not limited to, lactose, talc, silica, aluminum hydroxide, calcium silicate, or polyamide powder, which may be used alone or in combination. Particularly, the present invention made in a spray formulation may further include a propellant that may include, but is not limited to, chlorofluorohydrocarbon, propane/butane, or dimethyl ether, which may be used alone or in combination.

The carrier component available in the present invention prepared in a solution or emulsion formulation may be a solvent, solubilizer, or emulsifier, examples of which may include, but are not limited to, water, ethanol, isopropanol, ethyl carbonate, ethyl acetate, benzyl alcohol, benzyl benzoate, propylene glycol, or 1,3-butyl glycol oil; particularly, cottonseed oil, peanut oil, corn germ oil, olive oil, castor oil, or sesame seed oil; glycerol aliphatic esters; polyethylene glycols; or sorbitan fatty acid esters, which may be used alone or in combination.

The carrier component available in the present invention formulated as a suspension may include, but is not limited to, a liquid diluent, e.g., water, ethanol or propylene glycol; a suspending agent, e.g., ethoxylated isostearyl alcohol, polyoxyethylene sorbitol ester, or polyoxyethylene sorbitan ester; microcrystalline cellulose; aluminum metahydroxide; bentonite; agar; or tragacanth, which may be used alone or in combination.

The carrier component available in the present invention prepared in a soap formulation may include, but is not limited to, alkali metal salts of fatty acids, fatty acid hemiester salts, fatty acid protein hydrolysate, isethionate, lanolin derivatives, aliphatic alcohols, vegetable oils, glycerols, or sugars, which may be used alone or in combination.

In the cosmetic composition of the present invention, the three strains of Bacillus sp. pfbarrier 01 (KCTC 14628BP), Lactobacillus pentosus pfbio035 (KCTC 14684BP) and Lactobacillus sakei subsp. sakei pfbio005 (KCTC 14130BP), the culture solution thereof, or the compound extract thereof may be contained in an amount (dry-weight basis) of specifically 0.0001 to 50 wt. % and more specifically 0.0005 to 10 wt. % with respect to the total weight of the cosmetic composition. The content within the above-defined range is advantageous in that it can secure high effects of improving skin wrinkles, moisturizing skin, strengthening skin barriers, and promoting regeneration of wounded skin and stabilize the formulation of the composition.

In another aspect of the present invention, there is provided a food composition comprising three mixed bacterial strains of Bacillus sp. pfbarrier 01 (KCTC 14628BP), Lactobacillus pentosus pfbio035 (KCTC 14684BP) and Lactobacillus sakei subsp. sakei pfbio005 (KCTC 14130BP), a culture solution thereof, or a compound extract thereof as an active ingredient.

The three mixed bacterial strains of Bacillus sp. pfbarrier 01 (KCTC 14628BP), Lactobacillus pentosus pfbio035 (KCTC 14684BP) and Lactobacillus sakei subsp. sakei pfbio005 (KCTC 14130BP), the culture solution thereof, or the compound extract thereof according to the present invention can make high effects in improving skin wrinkles, moisturizing skin, strengthening skin barriers, and promoting regeneration of wounded skin. Hence, they can be beneficially used in the food composition for improving skin conditions. The three mixed strains of the present invention and their effects of improving skin wrinkles, moisturizing skin, strengthening skin barriers, and promoting regeneration of wounded skin are as specified above. The food composition may be used in the form of a health functional food, but is not limited thereto.

The food composition of the present invention may contain three mixed bacterial strains of Bacillus sp. pfbarrier 01 (KCTC 14628BP), Lactobacillus pentosus pfbio035 (KCTC 14684BP) and Lactobacillus sakei subsp. sakei pfbio005 (KCTC 14130BP), a culture solution thereof, or a compound extract thereof, or a processed product thereof. The composition may further include a foodologically acceptable food supplementary additive in addition to the active ingredients.

In the present invention, the term “food supplementary additive” means an auxiliary component added in the preparation of health functional foods in each formulation. Any one skilled in the art can appropriately select the food supplementary additive for use. Examples of the food supplementary additive may include, but are not limited to, a variety of nutrient supplements, vitamins, minerals (electrolytes), synthetic flavors, natural flavors, coloring agents, fillers, pectic acid and salts thereof, alginic acid and salts thereof, organic acids, protective colloidal thickeners, pH adjusters, stabilizers, preservatives, glycerin, alcohols, or carbonating agents for carbonated beverages.

The food composition of the present invention may include health functional foods. In the present invention, the term “health functional food” refers to a food prepared and processed in the form of tablets, capsules, powders, granules, liquids, or pills using raw materials or ingredients that have beneficial functions for human body. The term “functional” as used herein means regulating nutrients for the structure and functions of the human body or offering beneficial effects for purposes of health, such as physiological actions. The health functional food of the present invention can be prepared by a method commonly used in the related art and made with raw materials and ingredients commonly used in the preparation process in the related art.

Besides, the health functional food can be prepared in any type of formulation accepted as appropriate for health functional foods without any limitation. The health functional food of the present invention may be formulated as any one of various formulations. Unlike general drug products, it uses a mixture of edible lactic acid bacteria, causing no risk of side effects possibly occurring in the case of long-term consumption of foods. Further, it is highly portable and consumable as a supplement for enhancing the effects to improve skin wrinkles, moisturize skin, strengthen skin barrier, and promote regeneration of wounded skin.

The health functional food of the present invention may take any form without limitation and include any form of foods in the ordinary sense. The term “health functional food” can be used interchangeable with the terms known in the related art, such as “functional food”. Besides, the health functional food of the present invention may be prepared by appropriately mixing auxiliary ingredients and known additives available for foods by the choice of those skilled in the art. Examples of foods to which the present invention can be added may include meats, sausages, breads, chocolates, candies, snacks, cookies, pizzas, ramen and other noodles, gums, dairy products, such as ice cream, any type of soups, beverages, teas, drinks, alcoholic beverages, and vitamin complexes. The food may be prepared by adding the three mixed strains, the culture solution thereof, or the compound extract thereof according to the present invention as a main ingredient in liquid extracts, teas, jellies, or juices. It also includes foods used as feeds for animals.

The features and advantages of the present invention can be summarized as follows:

(1) The present invention provides a composition comprising three mixed bacterial strains of Bacillus sp. pfbarrier 01 (KCTC 14628BP), Lactobacillus pentosus pfbio035 (KCTC 14684BP) and Lactobacillus sakei subsp. sakei pfbio005 (KCTC 14130BP), a culture solution thereof, or a compound extract thereof as an active ingredient that exhibits beneficial activities in improving skin wrinkles, moisturizing skin, strengthening skin barriers, and promoting regeneration of wounded skin.

(2) The composition of the present invention poses non-cytotoxicity and high beneficial effects to improve skin wrinkles, moisturize skin, strengthen skin barriers, and help regeneration of wounded skin, which makes it highly applicable to the fields of foods and cosmetics.

(3) Besides, the composition of the present invention is not only very safe for the human body but also has high stability.

### BRIEF DESCRIPTION OF THE DRAWINGS

FIG. 1 presents graphs showing the cell viability of normal human dermal fibroblasts (NHDFs) after treatment with single-strain extracts of Bacillus sp. pfbarrier 01, Lactobacillus pentosus pfbio035 or Lactobacillus sakei subsp. sakei pfbio005, or a compound extract (pf-lacto) thereof.

FIG. 2 presents graphs showing the expression of matrix metalloproteinase-1 (MMP-1) gene in normal human dermal fibroblasts (NHDFs) after treatment with single-strain extracts of Bacillus sp. pfbarrier 01, Lactobacillus pentosus pfbio035 or Lactobacillus sakei subsp. sakei pfbio005, or a compound extract (pf-lacto) thereof.

FIG. 3 presents graphs showing the expression of type 1 procollagen gene in normal human dermal fibroblasts (NHDFs) after treatment with single-strain extracts of Bacillus sp. pfbarrier 01, Lactobacillus pentosus pfbio035 or Lactobacillus sakei subsp. sakei pfbio005, or a compound extract (pf-lacto) thereof.

FIG. 4 presents graphs showing the production yield of hyaluronan (hyaluronic acid) in human keratinocytes (HaCaT) after treatment with single-strain extracts of Bacillus sp. pfbarrier 01, Lactobacillus pentosus pfbio035 or Lactobacillus sakei subsp. sakei pfbio005, or a compound extract (pf-lacto) thereof.

FIG. 5 presents graphs showing the m-RNA expression of filaggrin in human keratinocytes (HaCaT) after treatment with single-strain extracts of Bacillus sp. pfbarrier 01, Lactobacillus pentosus pfbio035 or Lactobacillus sakei subsp. sakei pfbio005, or a compound extract (pf-lacto) thereof.

FIG. 6 presents graphs showing the regeneration rate of wounded skin after treatment with single-strain extracts of Bacillus sp. pfbarrier 01, Lactobacillus pentosus pfbio035 or Lactobacillus sakei subsp. sakei pfbio005, or a compound extract (pf-lacto) thereof.

### DETAILED DESCRIPTION OF THE EMBODIMENTS

Hereinafter, the present invention will be described in further detail with reference to examples, which are given only for illustration and construed to not limit the scope of the present invention.

### Example 1. Selection and Extraction of Strains

### 1-1. Selection of Strains

Soybeans and kimchi were collected, diluted with sterile saline and sprayed on BCP agar, to harvest colonies when the medium turned yellowish. The produced colonies were separately cultured in an MRS medium, and 16S rRNA sequencing was performed to identify the isolated strains. The 16S rRNA sequence of the isolated strains showed homologies of at least 99% to known strains, Bacillus subtilis IAM 12118, Lactobacillus pentosus DSM 20314 and Lactobacillus sakei MBEL 1397. The three strains thus isolated were named Bacillus sp. pfbarrier 01, Lactobacillus pentosus pfbio035 and Lactobacillus sakei subsp. sakei pfbio005 and deposited with the Korean Collection for Type Culture (KCTC) of the Korea Research Institute of Bioscience and Biotechnology under accession numbers of KCTC 14628BP, KCTC 14684BP and KCTC 14130BP, respectively.

### 1-2. Preparation of Strain Extracts

Each of Bacillus sp. pfbarrier 01 (KCTC 14628BP), Lactobacillus pentosus pfbio035 (KCTC 14684BP) and Lactobacillus sakei subsp. sakei pfbio005 (KCTC 14130BP) strains was inoculated into 100 ml of MRS broth (Difco) and cultured in an incubator (thermostatic chamber) at 37° C. until the OD₆₀₀ value became 1.0 (at OD 600 nm) to form a culture. 10 ml of the culture solution was centrifuged for 20 minutes at 4° C. and 4,000 rpm to collect the supernatant. The resultant culture solutions of the individual strains were freeze-dried into a culture extract of Bacillus sp. pfbarrier 01 (Preparation Example 1), a culture extract of Lactobacillus pentosus pfbio035 (Preparation Example 2) and a culture extract of Lactobacillus sakei subsp. sakei pfbio005 (Preparation Example 3) in powder form.

In addition, the individual single-strain extracts of the three bacterial strains were mixed in equal amounts to prepare a three-strain compound extract (Preparation Example 4).

### Example 2. Evaluation of Cytoprotective Effect Using Cytotoxicity Test (MTT Assay)

The single-strain extracts of Bacillus sp. pfbarrier 01 (KCTC 14628BP), Lactobacillus pentosus pfbio035 (KCTC 14684BP) or Lactobacillus sakei subsp. sakei pfbio005 (KCTC 14130BP) and the compound extract thereof as prepared in Example 1 were individually tested to determine their cytotoxicity to normal human dermal fibroblasts (NHDFs).

The cytotoxicity was measured using the MTT (3-(4,5-dimethylthiazol-2-yl)-2,5-diphenyltetrazolium bromide) colorimetric assay. The methyl thiazol tetrazolium (MTT) assay is used to measure cell viability, based on the reduction of MTT to a purple-colored formazan dye. Following 72 hours of incubation, the volume of the culture medium reduced to 1 ml. 100 μl of 1 mg/ml MTT was added into each well. Then, normal human dermal fibroblasts (NHDFs) were cultured for 2 hours at 37° C. in an atmosphere of 5% CO₂ and 95% O₂. The NHDFs were treated with the individual single-strain extracts or the compound extract prepared in Example 1 by the concentrations (50, 100, 250 μg/ml) and then incubated for 24 hours. Following 24 hours of incubation, 1 mg/ml MTT was added. In 3 hours, the formazan produced by the application of MTT on the cells was dissolved in DMSO to measure the absorbance at 570 nm.

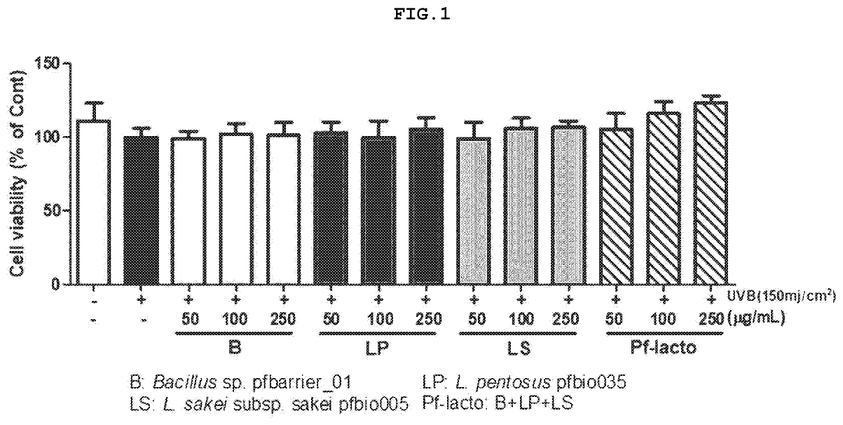

According to the experimental results, as shown in FIG. 1, the single-strain extracts of Bacillus sp. pfbarrier 01 (KCTC 14628BP), Lactobacillus pentosus pfbio035 (KCTC 14684BP) or Lactobacillus sakei subsp. sakei pfbio005 (KCTC 14130BP) had no toxicity to the normal human dermal fibroblasts (NHDFs). Plus, the test group treated with the three-strain compound extract (Preparation Example 4) had the cell proliferation increased by 105% (50 g/ml), 116% (100 g/ml) and 123% (250 μg/ml) in relation to the UV_{B}-irradiated (150 mJ/cm²) group. The bottom line is that the three-strain compound extract of the present invention poses no cytotoxicity and exhibits notably high cytoprotective effects, that is, promoting the growth of skin cells.

### Example 3. Skin Wrinkle Improving Activity

### 3-1. Inhibiting Expression of MMP-1 Gene

An analysis was performed to evaluate the effects of the individual single-strain extracts of Bacillus sp. pfbarrier 01, Lactobacillus pentosus pfbio035 or Lactobacillus sakei subsp. sakei pfbio005 and the compound extract thereof on the expression of MMP-1 gene.

2 ml of DMEM culture solution was put in a 40 mm cell culture dish. Normal human dermal fibroblasts (NHDFs) were seeded at a density of about 1.2×10⁵ cells/dish and cultured for 24 hours at 37° C. in an atmosphere of 5% CO₂. Then, the NHDFs were cultured for 3 days in a new culture medium containing the extracts of the culture solutions (50 μg/ml, 100 μg/ml and 250 g/ml) according to Preparation Examples 1 to 4 as prepared in Example 1. The resultant culture solutions were harvested and centrifuged for 5 minutes at 4° C. and 7, 500 rpm to determine the change in the expression of MMP-1 protein (Human Total MMP-1 kit, R&D Systems, Inc., Minneapolis, MN, USA) using the ELISA method. All the procedures were repeated three times.

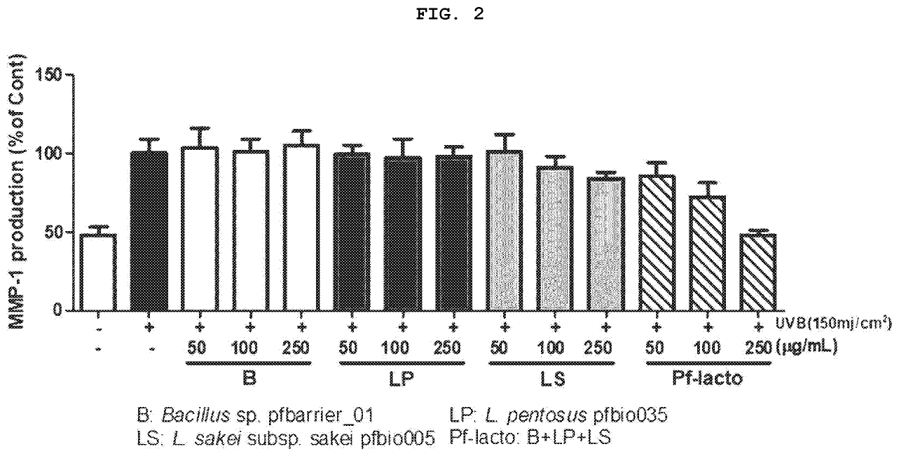

Referring to FIG. 2 presenting the experimental results in summary, the inhibitory effect against MMP-1 protein expression was highest in the test group treated with the culture extract of Lactobacillus sakei subsp. sakei pfbio005 out of the test groups treated with single-strain culture solutions. Besides, the test group treated with the three-strain compound extract of the present invention showed a more significant decrease in the production yield of the MMP-1 protein in a concentration-dependent manner than the test groups treated with the single-strain extracts.

Specifically, while the MMP-1 protein content (Con.) increased by UV_{B} irradiation (150 mJ/cm²) was taken as a reference, the test group treated with the single-strain extract of Bacillus sp. pfbarrier 01 had the MMP-1 protein expression of 103% (50 μg/ml), 101% (100 μg/ml) and 105% (250 μg/ml), displaying no significant difference in the MMP-1 protein expression in relation to the negative control group; the test group treated with the single-strain extract of Lactobacillus pentosus pfbio035 had the MMP-1 protein expression reduced by 1% (50 μg/ml), 3% (100 μg/ml) and 2% (250 μg/ml) in relation to the negative control group; and the test group treated with the single-strain extract of Lactobacillus sakei subsp. sakei pfbio005 had the MMP-1 protein expression reduced by 9% (100 g/ml) and 16% (250 μg/ml) in relation to the negative control group. The analytical results showed that the single-strain extracts other than the extract of Lactobacillus sakei subsp. sakei pfbio005 had made relatively insignificant effects in reducing the MMP-1 protein expression.

In contrast, the test group treated with the three-strain compound extract of the present invention had the protein expression of MMP-1 reduced by 15% (50 μg/ml), 28% (100 μg/ml) and 52% (250 μg/ml), suggesting that a combination of the three bacterial strains exerted synergistic effects to improve skin wrinkles through a significant reduction of the MMP-1 protein expression.

### 3-2. Increasing Expression of Type 1 Procollagen Gene

The procedures were performed to treat and culture the same samples under the same conditions as described in Example 3-1. Subsequently, the culture solutions were harvested and centrifuged for 5 minutes at 4° C. and 7,500 rpm to determine the change in the protein expression of type 1 procollagen (Procollagen Type I C Peptide EIA Kit, Takara, Shiga, Japan) using the ELISA method. All the procedures were repeatedly performed three times.

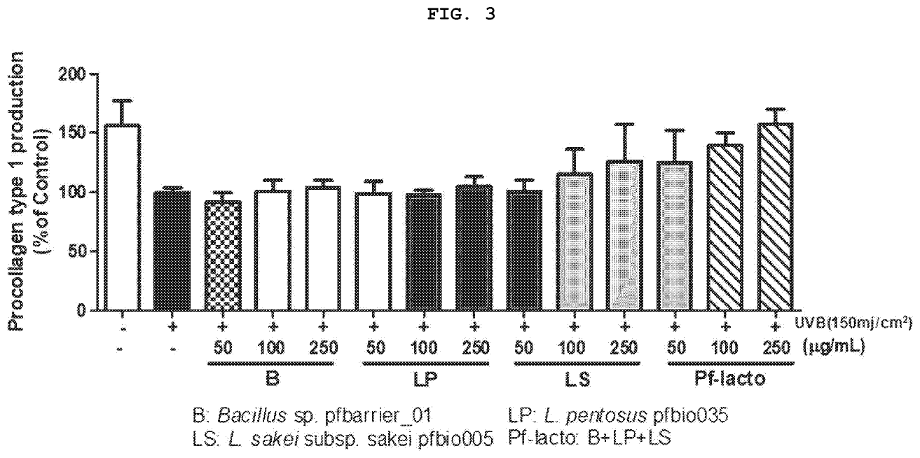

Referring to FIG. 3 presenting the experimental results in summary, the test group treated with the three-strain compound extract of the present invention had a significant increase in the protein expression of type 1 procollagen in a concentration-dependent manner in relation to the test groups treated with the single-strain extracts.

Specifically, while the protein expression (Con.) of type 1 procollagen reduced to two thirds by UV_{B} irradiation (150 mJ/cm²) was taken as a reference, the test group treated with the single-strain extract of Bacillus sp. pfbarrier 01 had the protein expression of type 1 procollagen increased by 101% (100 μg/ml) and 104% (250 μg/ml); the test group treated with the single-strain extract of Lactobacillus pentosus pfbio035 had the protein expression of type 1 procollagen increased by 105% (250 μg/ml); and the test group treated with the single-strain extract of Lactobacillus sakei subsp. sakei pfbio005 had the protein expression of type 1 procollagen increased by 101% (50 μg/ml), 115% (100 g/ml) and 126% (250 μg/ml). The analytical results showed that the single-strain extracts other than the extract of Lactobacillus sakei subsp. sakei pfbio005 showed relatively no effect in increasing the protein expression of type 1 procollagen.

In contrast, the test group treated with the three-strain extract of the present invention had the protein expression of type 1 procollagen increased by 125% (50 μg/ml), 139% (100 μg/ml) and 157% (250 μg/ml), indicating that a combination of the three bacterial strains exerted synergistic effects to improve skin a significant increase in the protein wrinkles through expression of type 1 procollagen.

As evaluated from the analytical results, the compound extract of the three bacterial strains of Bacillus sp. pfbarrier 01, Lactobacillus pentosus pfbio035 and Lactobacillus sakei subsp. sakei pfbio005 effectively inhibits the protein expression of MMP-1 and increases the protein expression of type 1 procollagen in skin cells, so it can be applied for use purposes associated with improvement of skin wrinkles.

### Example 4. Skin Moisturizing Activity

A change in the yield of hyaluronan synthesized with or without sample treatment was analyzed in order to verify the skin moisturizing effect of the single-strain extracts of Bacillus sp. pfbarrier 01, Lactobacillus pentosus pfbio035 or Lactobacillus sakei subsp. sakei pfbio005, and the compound extract thereof.

Hyaluronan (hyaluronic acid, or HA) is a substance that is predominantly produced by epidermal keratinocytes and dermal fibroblasts and binds with water to play an important role in creating an environment for cells to function normally. The reduced amount of hyaluronan in the skin is a direct cause of the decrease in skin elasticity and moisture content. Therefore, the experiment was performed to evaluate the yield of hyaluronan produced in human keratinocytes.

1 ml of the culture solution was put in a 24-well cell culture dish. Human keratinocytes (HaCaT) were seeded onto the culture dish at a density of about 1×10⁵ cells/dish and incubated for 24 hours at 37° C. in an atmosphere of 5% CO₂. Subsequently, the cells were incubated for 24 hours in a new culture medium containing the individual single-strain extracts of Bacillus sp. pfbarrier 01, Lactobacillus pentosus pfbio035 or Lactobacillus sakei subsp. sakei pfbio005 or the compound extract thereof as prepared in Example 1 (Preparation Examples 1 to 4) in amounts of 50, 100, 250 μg/ml. The culture solutions were harvested and centrifuged for 5 minutes at 4° C. and 7, 500 rpm, followed by using the ELISA method to determine the change in the expression amount of hyaluronan (Hyaluronan kit, R&D Systems, Inc., Minneapolis, MN, USA).

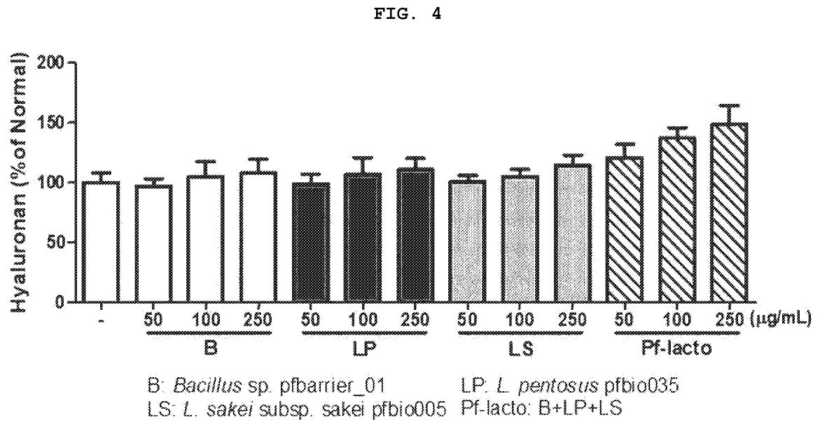

Referring to FIG. 4 presenting the experimental results in summary, the test groups treated with the individual single-strain extracts of the three bacterial strains or the three-strain compound extract thereof showed a tendency that the yield of hyaluronan increased in a concentration-dependent manner. Yet, there was a difference in the increase in the yield of hyaluronan between the test groups treated with the single-strain extracts and the test group with the compound extract. Such an increase in the yield of hyaluronan was notably higher in the test group treated with the compound extract.

Specifically, the test group treated with the three-strain compound extract of Bacillus sp. pfbarrier 01, Lactobacillus pentosus pfbio035 and Lactobacillus sakei subsp. sakei pfbio005 had the yield of hyaluronan increased by 121%, 137% and 149% at concentrations of 50, 100 and 250 μg/ml, respectively, in relation to the untreated group. This suggests that the three-strain compound extract made a higher synergistic effect in skin moisturization than the individual single-strain extracts.

As revealed from the results, the three-strain compound extract of the present invention effectively increases the production of hyaluronan in skin cells; hence, it can be very effectively applied for use purposes associated with skin moisturization.

### Example 5. Skin Barrier Strengthening Activity

An analysis was performed on the change in the expression of filaggrin (FLG, horny layer forming factor) induced by the single-strain extracts of Bacillus sp. pfbarrier 01, Lactobacillus pentosus pfbio035 or Lactobacillus sakei subsp. sakei pfbio005 or the compound extract thereof.

Firstly, human keratinocytes (HaCaT), used as a cell line, were seeded in a 100 mm cell culture dish at a density of 1×10⁶ cells/dish using DMEM medium supplemented with 10% FBS and cultured for 24 hours at 37° C. in a 5% CO₂ incubator. Following the incubation, a diluted solution prepared by diluting the single-strain extracts of the culture solutions of Bacillus sp. pfbarrier 01, Lactobacillus pentosus pfbio035 or Lactobacillus sakei subsp. sakei pfbio005 or the compound extract thereof with the DMEM medium to concentrations of 50, 100, 250 μg/ml was added for additional incubation, which was performed for 24 hours under the same conditions. Subsequently, the cells were harvested using a Trizol reagent (Invitrogen, USA) and removed of RNA. For RT-PCR, a PCR instrument (Step One Plus, Applied Biosystems, USA) was used, and a SYBR Green reagent (SYBR Green supermix, Applied Biosystems, USA) along with profilaggrin, GAPDH, and cDNA was added to activate polymerases for 5 minutes at 94° C., followed by 40 cycles of polymerization reaction for 30 seconds at 95° C., for one minute at 54° C. and for one minute at 72° C.

The primers used herein were given as follows:

Profilaggrin: Forward sequence
(SEQ ID NO: 1)
5′-AAG CTT CAT GGT GAT GCG AC-3′,
Reverse sequence
(SEQ ID NO: 2)
5′-TCA AGC AGA AGA GGA AGG CA-3′
GAPDH:
Forward sequence
(SEQ ID NO: 3)
5′-ACC ACA GTC CAT GCC ATC AC-3′,
Reverse sequence
(SEQ ID NO: 4)
5′-CCA CCA CCC TGT TGC TGT AG-3′

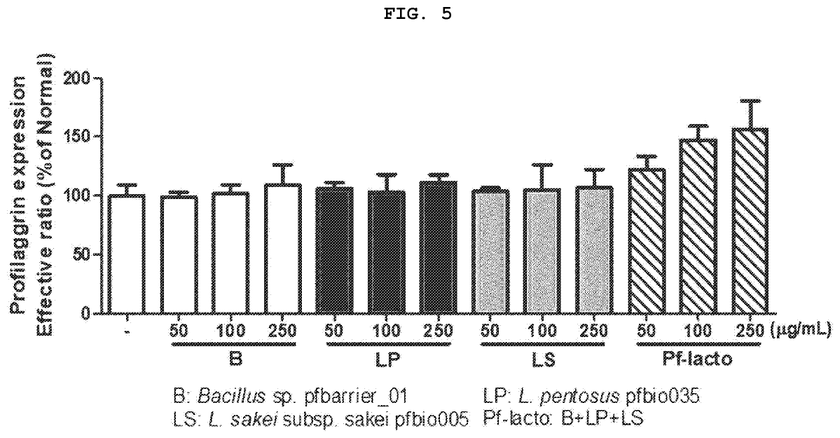

RT-PCR was performed using the above-stated primers. The experimental results are summarized in FIG. 5.

Filaggrin exists in the epidermal granular layer in form of profilaggrin, which is a protein constituting keratohyalin granules. Profilaggrin is broken down into filaggrin in the final differentiation process of keratinocytes. In formation of cornified cellenvelopes, the filaggrin aggregates keratin filaments to form a hard and flat structure of corneocytes and acts as a brick of the skin barrier.

Referring to FIG. 5 presenting the experimental results in summary, the test groups treated with the individual single-strain extracts of Bacillus sp. pfbarrier 01, Lactobacillus pentosus pfbio035 or Lactobacillus sakei subsp. sakei pfbio005 or the three-strain compound extract thereof showed an increase in the mRNA expression of profilaggrin in relation to the untreated group. Such an increase in the mRNA expression of profilaggrin was notably higher in the test group treated with the compound extract rather than in the test groups with the single-strain extracts.

Specifically, in relation to the untreated group, the test group treated with the single-strain extract of Bacillus sp. pfbarrier 01 had the mRNA expression of profilaggrin increased by 102% (100 μg/ml) and 109% (250 μg/ml); the test group treated with the single-strain extract of Lactobacillus pentosus pfbio035 had the mRNA expression of profilaggrin increased by 106% (50 μg/ml), 103% (100 μg/ml) and 111% (250 μg/ml); and the test group treated with the single-strain extract of Lactobacillus sakei subsp. sakei pfbio005 had the mRNA expression of profilaggrin increased by 104% (50 μg/ml), 105% (100 μg/ml) and 107% (250 g/ml). In contrast, the test group treated with the three-strain compound extract of the present invention had the mRNA expression of profilaggrin increased by 122% (50 μg/ml), 147% (100 μg/ml) and 156% (250 μg/ml) in relation to the untreated group. Such an increase in the mRNA expression of profilaggrin was notably higher in the test group treated with the compound extract rather than in the test groups with the single-strain extracts, demonstrating that the combination of the three bacterial strains resulted in such a synergistic effect.

As revealed from the analytical results, the compound extract of the three bacterial strains of Bacillus sp. pfbarrier 01, Lactobacillus pentosus pfbio035 and Lactobacillus sakei subsp. sakei pfbio005 increases the expression of profilaggrin protein and contributes to the formation of skin barriers, thereby exerting high effects in improving skin conditions.

### Example 6. Wounded Skin Regenerating Activity

This experiment was performed to verify the wounded skin regenerating effect of the single-strain extracts of Bacillus sp. pfbarrier 01, Lactobacillus pentosus pfbio035 or Lactobacillus sakei subsp. sakei pfbio005 or the compound extract thereof in a wounded skin model.

Specifically, normal human dermal fibroblasts (NHDFs) as skin cells were pre-cultured in a 100 mm cell culture dish until 80% confluency. Then, a sterile pipet tip was used to scratch the skin cells and create a “wound” area. The single-strain extracts of Bacillus sp. pfbarrier 01, Lactobacillus pentosus pfbio035 or Lactobacillus sakei subsp. sakei pfbio005 or the compound extract thereof were inoculated at concentrations of 50, 100 and 250 μg/ml. Following 48 hours of inoculation, the cell regenerating effect was evaluated.

In order to measure the cell proliferation, the skin cells were dyed using the methylthiazole-2yl-2,5-diphenyl tetrazolium bromide (MTT) analysis method and the Diff quick stain method and visually inspected with a microscope to determine the cell regeneration rate in relation to the untreated group.

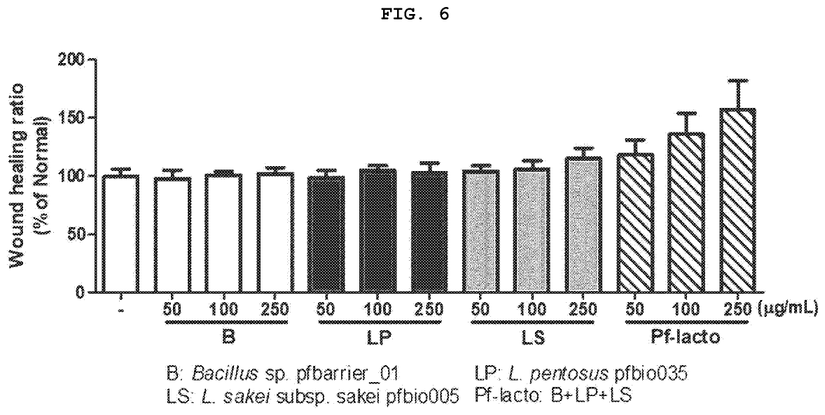

Referring to FIG. 6 presenting the experimental results in summary, the wounded skin cells upon treatment with the compound extract of Bacillus sp. pfbarrier 01, Lactobacillus pentosus pfbio035 and Lactobacillus sakei subsp. sakei pfbio005 grew to take the same morphological form of normal skin cells and exhibited an increase in the wounded skin regenerating activity.

The test groups treated with the single-strain extracts of Bacillus sp. pfbarrier 01, Lactobacillus pentosus pfbio035 and Lactobacillus sakei subsp. sakei pfbio005 or the compound extract thereof had an increase in the skin regenerating activity in relation to the untreated group. Skin regeneration appeared more notably in the test group treated with the compound extract rather than in the test groups with the single-strain extracts.

Specifically, in relation to the untreated group, the test group treated with the single-strain extract of Bacillus sp. pfbarrier 01 had the skin regeneration increased by 101% (100 μg/ml) and 102% (250 μg/ml); the test group treated with the single-strain extract of Lactobacillus pentosus pfbio035 had the skin regeneration increased by 105% (100 g/ml) and 103% (250 μg/ml); and the test group treated with the single-strain extract of Lactobacillus sakei subsp. sakei pfbio005 had the skin regeneration increased by 104% (50 μg/ml), 106% (100 μg/ml) and 115% (250 μg/ml). In contrast, the test group treated with the three-strain extract of the present invention had the skin regeneration increased by 118% (50 μg/ml), 136% (100 μg/ml) and 157% (250 μg/ml) in relation to the untreated group. Such an increase in the skin regeneration was notably higher in the test group treated with the compound extract rather than in the test groups with the single-strain extracts, demonstrating that the combination of the three bacterial strains made a synergistic effect.

As revealed from the analytical results, the compound extract of the three bacterial strains applied to the wounded skin exerts a synergistic effect to promote regeneration of the wounded skin without making any difference between the regenerated skin tissue and the surrounding normal skin tissues.

Though the foregoing description of the present invention has been presented on the specific parts of the present invention, it should be apparent to those skilled in the art that these specific descriptions are given merely as preferred embodiments and not to be construed as limiting the scope of the present invention. Accordingly, the substantial scope of the present invention is to be defined by the claims and their equivalents.

Bacillus sp. pfbarrier_01 strain was deposited in the Korea Research Institute of Bioscience and Biotechnology (having the address of 181, Ipsin-gil, Jeongeup-si, Jeolllabuk-do 56212, Republic of Korea) under the Access number of KCTC 14628BP on Jun. 29, 2021. The deposit has been made under the terms of the Budapest Treaty and all restrictions imposed by the depositor on the availability to the public of the biological material will be irrevocably removed upon the granting of a patent.

Lactobacillus pentosus pfbio035 strain was deposited in the Korea Research Institute of Bioscience and Biotechnology (having the address of 181, Ipsin-gil, Jeongeup-si, Jeolllabuk-do 56212, Republic of Korea) under the Access number of KCTC 14684BP on Aug. 26, 2021. The deposit has been made under the terms of the Budapest Treaty and all restrictions imposed by the depositor on the availability to the public of the biological material will be irrevocably removed upon the granting of a patent.

Lactobacillus sakei subsp. Sakei pfbio005 strain was deposited in the Korea Research Institute of Bioscience and Biotechnology (having the address of 181, Ipsin-gil, Jeongeup-si, Jeolllabuk-do 56212, Republic of Korea) under the Access number of KCTC 14130BP on Feb. 7, 2020. The deposit has been made under the terms of the Budapest Treaty and all restrictions imposed by the depositor on the availability to the public of the biological material will be irrevocably removed upon the granting of a patent.

### [Microorganism Deposit Receipt 1]

### Budapest Treaty on International Recognition of the Deposit of Microorganisms for the Purpose of Patent Procedure International Form

### Deposit Receipt

To Yang, Ji-Hye Receipt in the case of an Original Pf Nature, 90 Mayu-ro, 118 beon-gil, Deposit issued pursuant to Rule 7.1 Siheung-si, Gyeonggi-do, South Korea by INTERNATIONAL DEPOSITORY
AUTHORITY

I. IDENTIFICATION OF MICROORGANISM

| | |
|---|---|
| Identification reference given by | Accession number given by |
| DEPOSITOR: | INTERNATIONAL DEPOSITORY AUTHORITY: |
| Bacillus sp. pfbarrier_01 | KCTC 14628BP |

II. SCIENTIFIC DESCRIPTION AND/OR PROPOSED TAXONOMIC DESIGNATION
The microorganism identified under I above was accompanied by:
[ ] a scientific description
[ ] a proposed taxonomic designation
(Mark with a cross where applicable)
III. RECEIPT AND ACCEPTANCE
This INTERNATIONAL DEPOSITARY AUTHORITY accepts the microorganism
identified under I above, which was received by it on Jun. 29, 2021 (original
deposit date)¹⁾.
IV. RECEIPT OF REQUEST FOR CONVERSION
The microorganism identified under I above was received by this INTERNATIONAL
DEPOSITARY AUTHORITY and a request to convert the original deposit to a
deposit under the Budapest Treaty was received by it.
IV. INTERNATIONAL DEPOSITARY AUTHORITY

| | |
|---|---|
| Name: Korean Collection for Type | Signature (s) of person (s) having the |
| Cultures | power to represent the INTERNATIONAL |
| Address: Korea Research Institute of | DEPOSITARY AUTHORITY of authorized |
| Bioscience and Biotechnology (KRIBB) | official (s): |
| 183, Ipsin-gil Jeongeup-si, | Kim, Song-Gun (Director) |
| Jeollabuk-do, 56212, South Korea | Date: Jun. 29, 2021 |

Form BP/4 (KCTC form 17)

### [Microorganism Deposit Receipt 2]

### Budapest Treaty on International Recognition of the Deposit of Microorganisms for the Purpose of Patent Procedure International Form

### Deposit Receipt

To Yang, Ji-Hye Receipt in the case of an Original Pf Nature, 90 Mayu-ro, 118 beon-gil, Deposit issued pursuant to Rule 7.1 Siheung-si, Gyeonggi-do, South Korea by INTERNATIONAL DEPOSITORY
AUTHORITY

I. IDENTIFICATION OF MICROORGANISM

| | |
|---|---|
| Identification reference given by | Accession number given by |
| DEPOSITOR: | INTERNATIONAL DEPOSITORY AUTHORITY: |
| Lactobacillus pentosus pfbio035 | KCTC 14684BP |

II. SCIENTIFIC DESCRIPTION AND/OR PROPOSED TAXONOMIC DESIGNATION
The microorganism identified under I above was accompanied by:
[ ] a scientific description
[ ] a proposed taxonomic designation
(Mark with a cross where applicable)
III. RECEIPT AND ACCEPTANCE
This INTERNATIONAL DEPOSITARY AUTHORITY accepts the microorganism
identified under I above, which was received by it on Aug. 26, 2021
(original deposit date)¹⁾.
IV. RECEIPT OF REQUEST FOR CONVERSION
The microorganism identified under I above was
received by this INTERNATIONAL
DEPOSITARY AUTHORITY and a request to convert the original deposit to a
deposit under the Budapest Treaty was received by it.
IV. INTERNATIONAL DEPOSITARY AUTHORITY

| | |
|---|---|
| Name: Korean Collection for Type | Signature(s) of person(s) having the |
| Cultures | power to represent the INTERNATIONAL |
| Address: Korea Research Institute of | DEPOSITARY AUTHORITY of authorized |
| Bioscience and Biotechnology (KRIBB) | official(s): |
| 183, Ipsin-gil Jeongeup-si, | Kim, Song-Gun (Director) |
| Jeollabuk-do, 56212, South Korea | Date: Aug. 26, 2021 |

Form BP/4 (KCTC form 17)

### [Microorganism Deposit Receipt 3]

### Budapest Treaty on International Recognition of the Deposit of Microorganisms for the Purpose of Patent Procedure International Form

### Deposit Receipt

To Yang, Ji-Hye Receipt in the case of an Original Pf Nature, 90 Mayu-ro, 118 beon-gil, Deposit issued pursuant to Rule 7.1 Siheung-si, Gyeonggi-do, South Korea by INTERNATIONAL DEPOSITORY
AUTHORITY

I. IDENTIFICATION OF MICROORGANISM

| | |
|---|---|
| Identification reference given by | Accession number given by |
| DEPOSITOR: | INTERNATIONAL DEPOSITORY AUTHORITY: |
| Lactobacillus sakei subsp. Sakei | KCTC 14130BP |

pfbio005
II. SCIENTIFIC DESCRIPTION AND/OR PROPOSED TAXONOMIC DESIGNATION
The microorganism identified under I above was accompanied by:
[ ] a scientific description
[ ] a proposed taxonomic designation
(Mark with a cross where applicable)
III. RECEIPT AND ACCEPTANCE
This INTERNATIONAL DEPOSITARY AUTHORITY accepts the microorganism
identified under I above, which was received by it on Feb. 7, 2020
(original deposit date) 1).
IV. RECEIPT OF REQUEST FOR CONVERSION
The microorganism identified under I above was received by this INTERNATIONAL
DEPOSITARY AUTHORITY and a request to convert the original deposit to a
deposit under the Budapest Treaty was received by it.
IV. INTERNATIONAL DEPOSITARY AUTHORITY

| | |
|---|---|
| Name: Korean Collection for Type | Signature (s) of person (s) having the |
| Cultures | power to represent the INTERNATIONAL |
| Address: Korea Research Institute of | DEPOSITARY AUTHORITY of authorized |
| Bioscience and Biotechnology (KRIBB) | official(s) : |
| 183, Ipsin-gil Jeongeup-si, | Kim, Song-Gun (Director) |
| Jeollabuk-do, 56212, South Korea | Date: Feb. 7, 2020 |

Form BP/4 (KCTC form 17)

A sequence listing electronically submitted with the present application on Sep. 29, 2022 as an ASCII text file named 20220929_Q93922YG04_TU_SEQ. TXT, created on Sep. 7, 2022 and having a size of 930 bytes, is incorporated herein by reference in its entirety.

1: A cosmetic composition comprising mixed bacterial strains of Bacillus sp. pfbarrier 01 deposited under the accession number KCTC 14628BP, Lactobacillus pentosus pfbio035 deposited under the accession number KCTC 14684BP and Lactobacillus sakei subsp. sakei pfbio005 deposited under the accession number KCTC 14130BP, a culture solution thereof, or a compound extract thereof as an active ingredient.

2: The cosmetic composition of claim 1, wherein the composition inhibits the expression of matrix metalloproteinase-1 (MMP-1) gene and increases the expression of type 1 procollagen gene to improve skin wrinkles.

3: The cosmetic composition of claim 1, wherein the composition promotes the synthesis of hyaluronic acid to moisturize skin.

4: The cosmetic composition of claim 1, wherein the composition increases the expression of filaggrin gene to strengthen skin barrier.

5: The cosmetic composition of claim 1, wherein the composition promotes the regeneration of a wounded skin.

6: A food composition comprising mixed bacterial strains of Bacillus sp. pfbarrier 01 deposited under the accession number KCTC 14628BP, Lactobacillus pentosus pfbio035 deposited under the accession number KCTC 14684BP and Lactobacillus sakei subsp. sakei pfbio005 deposited under the accession number KCTC 14130BP, a culture solution thereof, or a compound extract thereof as an active ingredient.


SEQUENCE LISTING (4 sequences)

SEQ ID NO: 1 - DNA, 20 bp, Homo sapiens
aagcttcatggtgatgcgac

SEQ ID NO: 2 - DNA, 20 bp, Homo sapiens
tcaagcagaagaggaaggca

SEQ ID NO: 3 - DNA, 20 bp, Homo sapiens
accacagtccatgccatcac

SEQ ID NO: 4 - DNA, 20 bp, Homo sapiens
ccaccaccctgttgctgtag